(19) United States
(12) Patent Application Publication
(10) Pub. No.: US 2015/0051283 A1
(43) Pub. Date: Feb. 19, 2015

(54) COMPOSITION AND METHOD FOR INHIBITION OF PKNG FROM MYCOBACTERIUM TUBERCULOSIS

(71) Applicant: COMPLEXA, INC., Pittsburgh, PA (US)

(72) Inventors: Carlos Ignacio Batthyany Dighiero, Montevideo (UY); Rosario Duran, Montevideo (UY)

(21) Appl. No.: 14/306,191

(22) Filed: Jun. 16, 2014

Related U.S. Application Data
(60) Provisional application No. 61/835,416, filed on Jun. 14, 2013.

(51) Int. Cl. A61K 31/201

(52) U.S. Cl. CPC A61K 31/201
USPC 514/560

(57) ABSTRACT

PknG from Mycobacterium tuberculosis is a Ser/Thr protein kinase that regulates key metabolic processes within the bacterial cell as well as signaling pathways from the infected host cell. This multi-domain protein has a conserved canonical kinase domain with N- and C-terminal flanking regions of unclear functional roles. The N-terminus harbors a rubredoxin-like domain (Rbx), a bacterial protein module characterized by an iron ion coordinated by four cysteine residues. Disruption of Rbx-metal binding site by simultaneous mutations of all the key cysteine residues significantly impairs PknG activity. This encouraged us to evaluate the effect of a nitro-fatty acid (9- and 10-nitro-octadeca-9-cis-enoic acid; OA-NO₂) on PknG activity. Fatty acid nitroalkenes are electrophilic species produced during inflammation and metabolism that react with nucleophilic residues of target proteins (i.e. Cys and His), modulating protein functions and subcellular distribution in a reversible-manner. In accordance with the present invention, administration of OA-NO₂ inhibits kinase activity by covalently adducting PknG outside its catalytic domain. Mass spectrometry-based analysis established that cysteines located at Rbx are the specific targets of the nitroalkene. Cys-nitroalkylation is a Michael addition reaction typically reverted by thiols. However, the reversible OA-NO₂ mediated nitroalkylation of the kinase results in an irreversible inhibition of PknG. Cys adduction by OA-NO₂ induced iron release from the Rbx domain, revealing a new strategy for the specific inhibition of PknG. Altogether our results highlight the relevance of Rbx domain as an interesting new target for PknG inhibition. In addition, the reactivity of electrophilic fatty acids towards Rbx-Cys points to its potential as modulators of critical cell signaling activities and as model molecules for specific PknG inhibitors development.

### CROSS-REFERENCE TO RELATED APPLICATIONS

This application claims priority to co-pending U.S. Patent Provisional Application Ser. No. 61/835,416 filed Jun. 14, 2013, which is herein incorporated by reference in its entirety.

### BACKGROUND OF THE INVENTION

Mycobacterium tuberculosis, the causative agent of tuberculosis, is a major public health problem that causes more than one million deaths every year (See, e.g., http://goo.gl/A6X0i). Several factors comprise the efficacy of the available pharmacological treatments, including the emergence of multi- and extensive-drug resistant strains, the lack of new drugs, and the bacilli's ability to persist inside the host macrophages by inhibiting phagosome maturation. One of the most promising strategies to face with the urgency of drug discovery in tuberculosis disease is to interfere with bacterial signaling cascades and host cell transduction pathways [1, 2]. (Citations to reference notations in bracketing “[ ]” are found under the Reference section of the present application, infra).

Genome studies uncovered eleven Ser/Thr protein kinases akin to eukaryotic ones [3]. Among them, PknG, has emerged as a key soluble kinase that regulates critical processes in mycobacterial pathophysiology [1, 4, 5]. Experimental data support different functional roles for PknG including regulation of metabolic processes and interference of signaling pathways from the infected host cell [4, 5]. The inactivation of the pknG gene decreases cell viability and virulence in animal models, and suggests its participation in the control of intracellular glutamate/glutamine levels [6]. Our previous results revealed that PknG participates in the regulation of glutamate metabolism via the phosphorylation of an endogenous substrate (GarA), and the same function was reported for PknG from the related actinomycete Corynebacterium glutamicum [5, 7]. It has also been reported that PknG is a virulence factor that mediates M. tuberculosis ability to survive within host cells. Inside macrophages the kinase prevents phagosome-lysosome fusion by a still unknown mechanism [4]. In addition, inhibition of PknG activity yield bacteria more susceptible to intracellular degradation[4]. Due to the key cellular processes that it controls, PknG inhibition has emerged as an attractive strategy for potential drug discovery. The main challenge to overcome is to achieve selectivity for PknG inhibition, as the catalytic mechanisms as well as active Ser/Thr protein kinase fold is remarkable conserved form prokaryotes to eukaryotes.

PknG from M. tuberculosis is a multi-domain protein. The conserved canonical catalytic kinase domain is flanked by N- and C-terminal domains having undefined functional roles. The C-terminal domain of PknG contains a tetratricopeptide repeats motif (TPR), a domain known to participate in protein-protein interactions in both eukaryotic and prokaryotic organisms [8]. TPR domain is involved in intermolecular interactions in the reported crystal structure of PknG, however, whether and how dimerization is linked to enzyme activity is currently unknown [8]. The N-terminal sequence preceding the kinase domain contains both the autophosphorylation sites and a rubredoxin-like domain (Rbx) [5]. This protein module is typified by an iron ion coordinated by four conserved cysteine residues, and it was reported to participate in electron transfer reactions [9, 10]. The N-terminal sequence of PknG contains two CXXCG (Seq. No. 12) motifs typically involved in metal binding in Rbx domains. The crystal structure of a PknG construct confirmed the presence of a Rbx-like arrangement interacting with the kinase domain without occluding active site accessibility [8]. The role of Rbx domain in PknG is still uncertain. Metal binding site disruption by simultaneous mutations of multiple key cysteine residues has a remarkable effect on PknG activity [8, 11], pointing to a relevant functional or structural role of Rbx domain. This finding encouraged us to evaluate the effect of an electrophilic-nitrated fatty acid on PknG activity.

### SUMMARY OF THE INVENTION

Electrophilic unsaturated fatty acid derivatives generated by metabolic processes are emerging as endogenous signaling mediators that induce anti-inflammatory and chemotherapeutic responses [12]. In particular, nitrated unsaturated fatty acids are potent electrophiles that mediate the reversible nitroalkylation of specific proteins at nucleophilic Cys and His residues. This thiol-reversible post-translational modification modulates protein function and distribution [13]. The reactivity of these molecules is directed by the electrophilic carbon β to the electron-withdrawing NO₂ group, allowing reversible Michael addition with nucleophilic amino acids [12-14]. Compared with other biological electrophilic lipids, nitro-fatty acids (NO₂-FA) react with thiols with a high rate constant [15]. Moreover, the reaction between NO₂—FA and nucleophilic amino acids is also unique in that adduction reactions are thiol-reversible [12, 13].

It has been an objective of the present invention to exploit unique structural features of PknG to inhibit its kinase activity by a specific modification of its non-catalytic Rbx domain.

It is a further objective of the present invention to elucidate a new mechanism for kinase inhibition by iron release from the Rbx domain upon cysteine covalent modification by nitrated fatty acids.

### BRIEF DESCRIPTION OF THE FIGURES

FIGS. 1A and 1B depicts that Rbx and kinase domain co-occurrence is restricted to few Actinomycetales. FIG. 1A: Distance-based tree of 652 sequence homologs with pairwise identities <76%. The sequences were aligned with Mafft [22] and the tree built with BioNJ [38]. Zoom in the Glade containing sequences harboring both kinase and Rbx domains. They were realigned with T-Coffee [23] for a maximum-likelihood reconstruction with PhyML [26]. The two non actynobacterial sequences (A. cellulolyticus and K. racemifer) were omitted. FIG. 1B: Maximum-likelihood phylogenetic reconstruction for a set of 52 dissimilar homologous proteins. Species names and accession numbers are shown between underscores. Branch support values are displayed in red, omitting those from more internal nodes for clarity. The bottom scale bar indicates average substitutions per site.

FIG. 2: illustrates the overall fold of PknG kinase and rubredoxin domains. The catalytic domain of M. tuberculosis PknG (PDB code 2PZI) shown in ribbon representation (colored from blue to red) interacts with the Rbx domain (ribbon+surface representation). The four Cys residues of the Rbx domain and the bound metal (red) are shown. The figure was drawn with Pymol.

FIG. 3: illustrates the structure of nitrated oleic acid (OA-NO₂). Two regioisomers of OA-NO₂ were synthesized by nitrosenylation of oleic acid yielding 9- and 10-nitro-9-cis-octadecenoic acids. Taken from Baker, P. R., et al., “Fatty acid transduction of nitric oxide signaling: multiple nitrated unsaturated fatty acid derivatives exist in human blood and urine and serve as endogenous peroxisome proliferator-activated receptor ligands.” The Journal of Biological Chemistry 280:42464-42475; 2005.

FIGS. 4A-4D are a series of graphs depicting PknG kinase activity inhibition by OA-NO₂, in which FIG. 4A depicts GarA phosphorylation by PknG by linear MALDI-TOF mass spectrum of: GarA (m/z 17145, dashed line), GarA phosphorylated by PknG using a molar ratio PknG:GarA of 1:20 (m/z 17222, grey line) and GarA phosphorylated by PknG pretreated with 50 μM OA-NO₂ for 10 minutes (m/z 17143, black line), FIG. 4B is a series of three panels depicting PknG autophosphorylation with an upper panel illustrating linear MALDI-TOF spectrum of native PknG after tryptic digestion showing basal autophoshporylation pattern. Unphosphorylated peptide (m/z 5395.8), monophosphorylated (m/z 5475.4) and diphosphorylated species (m/z 5555.1) are detected as expected, a middle panel depicting linear MALDI-TOF spectrum of PknG after incubation with ATP and Mn²⁺ and further typtic digestion (autophoshorylation positive control). Diphosphorylated peptide (m/z 5555.0) is the most intense ion observed while unphosphorylated and monophosphorylated ions were almost undetectable, and a lower panel depicting linear MALDI-TOF spectrum of tryptic digestion of PknG treated with 30 μM OA-NO₂ for 10 min. before autophosporylation reaction. The detection of all three forms of the phosphorylatable peptide indicates that OA-NO₂ impairs their conversion into the fully phosphorylated species. The spectra depicted in the upper, middle and lower panels are each representative of three independent experiments; FIG. 4C depicts dose dependent inhibition of PknG by OA-NO₂ in which PknG (8 μM) was incubated with OA-NO₂ ranging from 0 to 80 μM in 70 mM ammonium bicarbonate, pH 8.0 at 25° C. As a control, PknG was incubated with vehicle under the same experimental conditions. After 10 minutes of incubation enzyme activities were determined. Samples were analyzed in triplicates; and FIG. 4D depicts time dependent inhibition of PknG by OA-NO₂. PknG (10 μM) was incubated with 50 μM OA-NO₂ (treated PknG) or vehicle (control PknG) in 70 mM ammonium bicarbonate, pH 8.0 at 25° C. At the indicated time points aliquots of control and treated PknG were removed, and enzyme inhibition was determined using Kinase Glo® by comparison with control activity. Samples were analyzed in triplicates.

FIGS. 5A-5H are a series of spectral graphs depicting modification sites of PknG by OA-NO₂ in which FIGS. 5A-5C are a series of panels illustrating MALDI-TOF mass spectra of Cys containing peptides generated by tryptic digestion of untreated PknG. FIG. 5A: peak m/z 1292.56 corresponds to seq. FC₁₀₆WNC₁₀₉GRPVGR (Seq. No. 2) with an intra molecular disulphide bridge. FIG. 5B: peak m/z 3530.62 corresponds to seq. GASEGWC₁₂₈PYC₁₃₁GSPYSFLPQLNPGDIVAGQYEVK (Seq. No. 3) with an intra molecular disulphide bridge. FIG. 5C: peak m/z 1813.92 (GC₁₅₆IAHGGLGWIYLALDR) (Seq. No. 13) is indicated, FIGS. 5D-5F are a series of panels depicting MALDI-TOF mass spectra of Cys containing peptides generated by tryptic digestion of OA-NO₂-treated PknG. FIG. 5D: peak m/z 1292.57 is partially consumed and a new peak appears showing a mass increment of 198/200/202 Da. Inset. Zoom in showing the 198/200/202 pattern and isotopic distribution. FIG. 5E: peak m/z 3530.61 is also partially consumed and again a new signal appears showing a mass increment of 198/200/202 Da. Inset. Zoom in showing the 198/200/202 mass increment pattern. FIG. 5F: Signal intensity of m/z 1813.93 is unchanged upon treatment with OA-NO₂, and in agreement with this observation we could not detect new signals in this m/z range (the arrows indicate the expected m/z values: +327 for OANO₂ modification and +198/200/202 for the previously experimentally observed mass shift), FIGS. 5G and 5H are a MALDI-TOF MS/MS spectrum of ion at m/z 3730.60 obtained from tryptic digestion of OA-NO₂-treated PknG. FIG. 5G: Sequence No. 3 showing main observed fragments (natives or modified). FIG. 5H: The spectra are representative of five independent experiments.

FIGS. 6A-6F are a series of panels depicting the effects of treatment of isolated Cys-containing peptides from PknG with OA-NO₂. FIGS. 6A-6C: MALDI-TOF mass spectrum of peptides generated by tryptic digestion of native PknG. Peptides were purified by reverse-phase chromatography prior to MS analysis. FIG. 6A: peak m/z 1294.63 (FC₁₀₆WNC₁₀₉GRPVGR) (Seq. No. 2), FIG. 6B: peak m/z 1815.04 (GC₁₅₆IAHGGLGWIYLALDR) (Seq. No. 13) and FIG. 6C: peak m/z 3532.80 (GASEGWC₁₂₈PYC₁₃₁GSPYSFLPQLNPGDIVAGQYEVK) (Seq. No. 3). The reduced peptides containing de CXXCG (Seq. No. 12) sequence are detected after the chromatographic step but are spontaneously converted into the intermolecular disulphide bridged oxidized form. FIGS. 6D-6F: MALDI-TOF mass spectrum of isolated peptides treated with OA-NO₂. FIG. 6D: a new peak appears showing a mass shift of 198/200/202 Da relative to m/z 1292.63, FIG. 6E: a new peak appears showing a mass shift of 196/198/200 Da relative to m/z 1814.94 and FIG. 6F: a new peak appears showing a mass shift of 198/200/202 Da relative to m/z 3530.61.

FIGS. 7A-7C are a series of panels illustrating MS/MS spectra of OA-NO₂ modified peptide Seq. No. 13. FIG. 7A: MALDI ionization. MS/MS spectrum of ion at m/z 2014.31 (native peptide+200 Da) obtained using MALDI-TOF MS. FIG. 7B: ESI ionization. MS/MS spectrum of ion at m/z 2141.85 (native peptide+327 Da) obtained using LC-MS. FIG. 7C: List of theoretical m/z values of fragment ions from peptide of m/z 2141.85 (GC₁₅₆IAHGGLGWIYLALDR (Seq. No. 13) modified by OA-NO₂). y- and b-ions detected by LC-MS are highlighted in bold.

FIGS. 8A-8H: Cysteine reactivity against other alkylating agents. PknG was exposed to IAM as a model reagent for cysteine alkylation. FIGS. 8A-8C: Mass spectrum of tryptic digest of native PknG. FIG. 8A: peak m/z 1292.58 Seq. No. 2 is shown, FIG. 8B: peak m/z 3530.59 Seq. No. 3 is indicated and FIG. 8C: peak m/z 1814.94 Seq. No. 13 is indicated. FIGS. 8D-8F: Mass spectrum of peptides obtained from tryptic digestion of PknG (10˜M) exposed to IAM (150˜M). FIG. 8D: no new signal appeared in the spectra, FIG. 8E: no modification appeared in the spectra FIG. 8F: after the treatment with IAM a new peak showing a mass increase of 57 Da appeared. FIG. 8G: Fragmentation pattern of m/z 1871.95. The increment of 57 Da in ion fragments corresponds to the incorporation of IAM. FIG. 8H: Mass spectrum of: native GarA (upper panel), GarA phosphorylated by native PknG (middle panel) and GarA phosphorylated by PknG previously treated with IAM (lower panel).

FIGS. 9A-9H: Identification of modified histidine residues on nitroalkylated PknG. PknG was exposed to OA-NO₂ in a molar ratio PknG:OA-NO₂ of 1:5. FIG. 9A: mass spectrum of peptides generated by tryptic digestion of native PknG, peak m/z 732.39 Seq. No. 8 (HFTTAR) is indicated. FIG. 9B: mass spectrum of peptides generated by tryptic digestion of PknG treated with OA-NO₂. FIG. 9C: Fragmentation pattern of modified peptide Seq. No. 8 obtained from tryptic digest of OA-NO₂-treated PknG. H₆₃₅ was identified as the modified residue. FIG. 9D: List of MS/MS fragment ions m/z of OA-NO₂ modified peptide 635-640. Detected ions are highlighted in bold. FIG. 9E: mass spectrum of peptides generated by tryptic digestion of native PknG, peak m/z 1800.90 (H₅₅₈FTEVLDTFPGELAPK) (Seq. No. 7), m/z 1843.86 (GLVH₁₈₅SGDAEAQAMAMAER) (Seq. No. 4) and m/z 1854.95 (ASTNH₇₀₂ILGFPFTSH711GLR) (Seq. No. 9) are indicated. FIG. 9F: mass spectrum of peptides generated by tryptic digestion of PknG treated with OA-NO₂. FIG. 9G: mass spectrum of peptides generated by tryptic digestion of native PknG, peak m/z 2486.19 (H₄₈₈GALDADGVDFSESVELPLMEVR) (Seq No. 6) and m/z 2614.30 (STFGVDLLVA₄₃₀TDVYLDGQV₄₄₀AEK) (Seq. No. 5) are indicated. FIG. 9H: mass spectrum of peptides generated by tryptic digestion of PknG treated with OA-NO₂.

FIGS. 10A and 10B: Modification sites of PknG 474/TPR by OA-NO₂. FIG. 10A: mass spectrum of tryptic digest of native PknG 474/TPR, cysteine-containing peptide (GASEGWC₁₂₈PYC₁₃₁GSPYSFLPQLNPGDIVAGQYEVK) (Seq. No. 3) is indicated. FIG. 10B: mass spectrum of tryptic digest of OA-NO₂-treated PknG Δ74/TPR. A new signal appears showing a mass increment of 198/200/202 Da with respect to peak m/z 3530.63.

FIGS. 11A and 11B depict irreversible PknG inhibition by reversible OA-NO₂ mediated nitroalkylation.

FIG. 11A: PknG was inhibited by incubation with OA-NO₂ (50 μM) in 70 mM ammonium bicarbonate, pH 8.0 at 25° C. for 10 min, as previously. Kinase activity of PknG inhibited by OA-NO₂ was measured (PknG+OA-NO₂). Subsequently, nitroalkylated samples were treated with DTT (42 mM) or GSH (24 mM) for 15 min at 25° C. and kinase activity was re-measured (PknG+OA-NO₂+RSH). Activity recovery was expressed as a ratio between [activity of PknG+OA-NO₂+RSH]/[activity PknG+OA-NO₂] where 1 means no activity recovery after thiol-containing agent treatment. GAPDH was used as a positive control: after DTT treatment 90% of the initial activity was recovered representing a 12 fold increase in the enzyme activity after the RSH treatment. Treatment of control PknG samples with 42 mM DTT or 24 mM GSH showed that these concentrations of the thiol reagents had no effect on kinase activity per se. Three independent experiments were performed FIG. 11B: MALDI-TOF mass spectrum of peptides generated by tryptic digestion of PknG. Upper panel: untreated PknG, middle panel: PknG exposed to OA-NO₂ and lower panel: idem middle panel+42 mM DTT. In each panel the native or nitroalkylated form of the peptide with the sequence 122-154 that includes the Rbx-Cys 128 and 131, are shown. All the spectra are representative of three independent experiments.

FIG. 12 depicts that OA-NO₂ induces iron release from Rbx domain. Non protein-bound iron present in control and OA-NO₂ treated PknG samples was recovered, reduced with DTT and quantified as Fe²⁺ using bathophenantrolinedisulfonic acid. The complex absorbs at 535 nm (λ_{535 nm} [BPS·Fe²⁺]=22140 M⁻¹ cm⁻¹ [17]). Iron present on native PknG samples (dashed line) or from OA-NO₂-treated PknG (solid line) are shown.

FIGS. 13A and 13B illustrate that OA-NO₂ modification does not generate a global change in PknG structure. FIG. 13A: Fluorescence of PknG-ANS complexes was collected using excitation wavelength set on 350 nm and emission between 370-650 nm. PknG natively folded is represented as solid black line; PknG exposed to OA-NO₂ is shown as solid grey line and PknG thermally denatured is indicated as dashed black line. FIG. 13B: Far UV-CD spectra of OA-NO₂-treated PknG (black circles) and OA-treated PknG (white diamonds).

### DETAILED DESCRIPTION OF THE PREFERRED EMBODIMENTS

As used herein, the following abbreviations have the following meanings: ANS, 8-anilino-1-naphthalenesulfonic acid ammonium salt; BPS, bathophenanthroline disulfonate; ESI, electrospray ionization; IAM, iodoacetamide; IPTG, isopropyl β-D-1-thiogalactopyranoside; LC, liquid chromatography; OA oleic acid, 9-octadecenoic acid; OA-NO₂ nitro-oleic acid, 9- and 10-nitro-9-cis-octadecaenoic acids; Rbx, rubredoxin.

The analysis of Mycobacterium tuberculosis genome sequence predicted the presence of eleven eukaryotic like Ser/Thr protein kinases denominated pknA to pknL [3]. However, the importance of these enzymes in mycobacterial physiology and virulence has been realized only recently [1, 32]. In particular, one of these enzymes, PknG, participates in the regulation of critical biological processes of M. tuberculosis. Previous data demonstrated that PknG regulates glutamate metabolism in M. tuberculosis through the phosphorylation of GarA, an intermediate regulator of three metabolic enzymes [5, 33]. PknG has also been proposed as an important virulence factor that contributes to inhibition of phagosomes maturation of infected macrophages through a still unknown mechanism [4, 11]. In agreement with this observation, inhibition of PknG activity yield bacteria more susceptible to degradation inside macrophages.

In this scenario, novel and selective PknG inhibitors could represent promising molecules for drug development. Despite research efforts, few PknG inhibitors displaying moderate activity have been already described [2, 8, 34]. The reported inhibitors are directed towards the kinase catalytic site, in the case of inhibitor AX20017 some degree of specificity towards PknG has been reported based on particular structural features of its active site [8]. It is well recognized that the active “eukaryotic like” kinase folding is highly conserved even among different kingdoms [35]. This turns specificity a difficult task to achieve. An alternative strategy for particular kinase inhibition contemplated by the present invention is to target other protein domains besides active site.

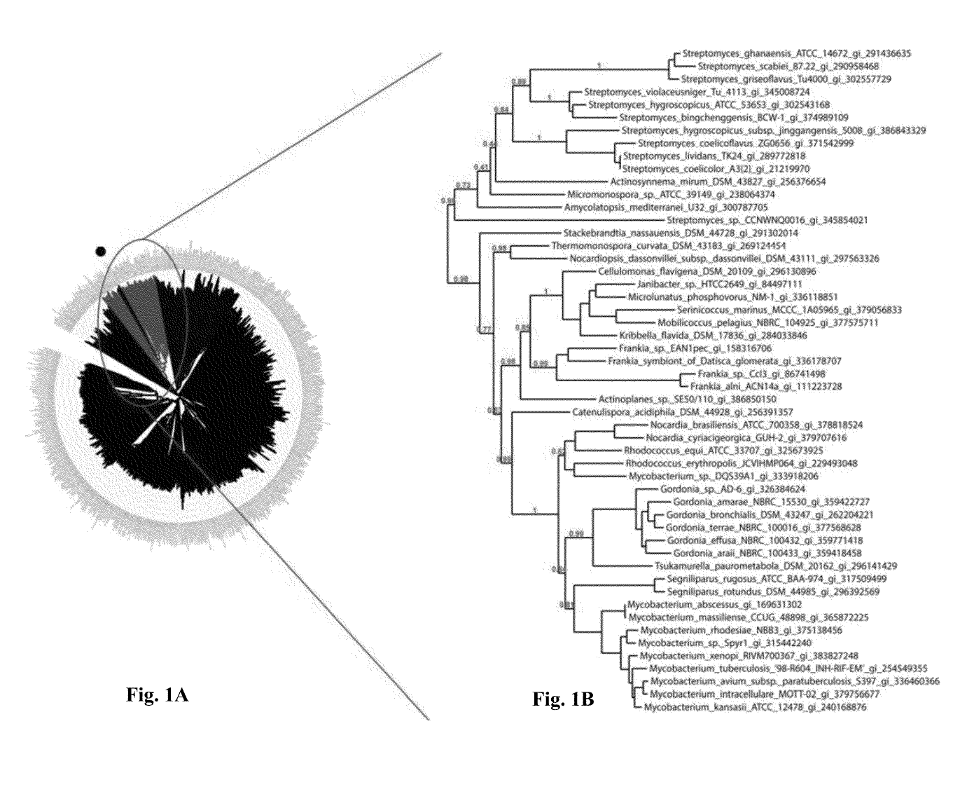

In one embodiment of the present invention, OA-NO₂ is used to inhibit PknG kinase activity by reversible alkylation of specific Cys residues of the Rbx domain, outside the catalytic domain. The combination of Rbx and kinase domains has not been previously been described for any other protein besides PknG-like kinases. PknG orthologs are present in all mycobacterial genomes sequenced to date as well as in other related actinomycetes [31]. Interestingly, while the kinase domain is very well preserved, some other domains are not. In particular, the N-terminal Rbx domain appears in few PknG-like kinases from Actinomycetales (FIG. 1). Moreover, PknG ortholog from Corynebacterium spp, with the equivalent function on glutamate metabolism, lacks the Rbx domain. Accordingly, another embodiment of the present invention includes nitrated fatty acids as selective inhibitors towards a subset of Rbx-containing enzymes homologous to PknG among prokaryotic and eukaryotic kinases.

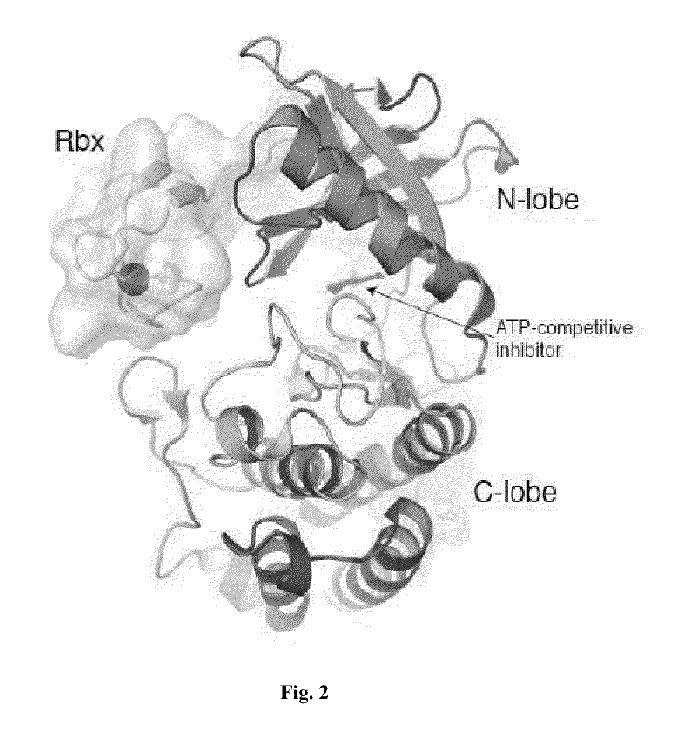

The structure of a truncated form of PknG shows that the rubredoxin-like domain is closely associated to the N-terminal lobe of the catalytic domain of PknG, just facing the kinase active site (FIG. 2). Thus, the modification of the Rbx domain could directly affect the active kinase conformation. In one embodiment of the present invention, the modification of Rbx-Cys by the nitroalkene is reversible but leads to a permanent effect on the enzyme activity because of the concomitant loss of the iron atom. Nitroalkylation of PknG did not induce a global change in its structure (FIGS. 13A-13B). In agreement with this observation, it has been demonstrated that the conversion of the well characterized bacterial rubredoxins to its apo form is followed by minor structural changes [28]. Without wishing to be bound by theory, minor local structural changes and/or the loss of iron per se, mediate a regulatory role of kinase activity. Previous attempts to generate apo-Rbx-PknG under not denaturizing conditions and to re-incoporate iron ion on OA-NO₂ treated PknG did not allow reaching more definitive conclusions. Elucidating the effect of iron release on Rbx structure and function will require further investigation. It is interesting to note that PknG is one of the two soluble kinases present in Mycobacterium tuberculosis lacking an extracellular sensor domain. The signals that activate/inactivate PknG and the mechanism for activity regulation are still completely unknown. The possible role of Rbx domain and iron ion on activity regulation deserves further investigation.

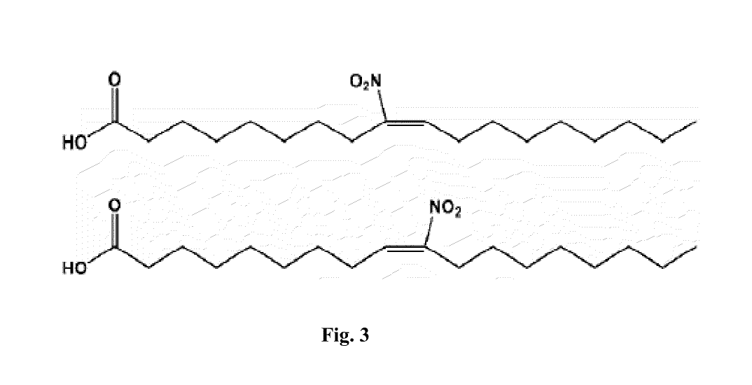

Fatty acid nitroalkenes are electrophilic species produced during inflammation and metabolism that react with nucleophilic amino acid residues of target proteins (i.e. Cys and His residues), modulating protein functions and their subcellular distribution in a reversible-manner. Nitroalkene reactivity is mainly directed by the electrophilic character of the β-carbon proximal to the alkenyl NO₂ group (FIG. 3) [16]. Nitrated fatty acids react with nucleophilic residues via a Michael addition-like mechanism to generate covalent adducts, that can be reverted by thiol-containing molecules rendering the native protein with its original function[13]. Rbx-Cys are classically reported as poor nucleophiles that do not react with regular alkylating reagents. However, mass spectrometry-based analysis confirmed that cysteines located at Rbx domain are the specific targets of the nitroalkene during its reaction with PknG. Even when the kinase sequence contains a free reactive Cys and many His, the present invention contemplates a totally new reactivity of nitroalkenes towards Rbx-Cys. Without wishing to be bound by theory, the present invention achieves irreversible inhibition by a reversible nitroalkylation of a redox-sensitive non-catalytic domain.

Xanthine oxidoreductase (XOR) inhibition by OA-NO₂ was the first irreversible inhibition reported for this nitroalkene. As in the case of PknG, XOR inhibition is not reversed by thiol reagents [15]. At that time, the postulated inhibition of XOR was mediated via: 1) an irreversible covalent reaction between OA-NO₂ and XOR or 2) a reaction of the nitroalkene with the dithiolene of the pterin moiety and the concomitant loss of the molybdenum atom; but no direct evidence was available at that time for any of the two hypotheses [15]. Inhibition of other enzymes by nitrated fatty acids was also previously reported [13, 36]. OA-NO₂ inhibition of both GAPDH and PknG is achieved at almost the same range of micromolar concentrations. It is important to note that OA-NO₂ is consider a potent inhibitor of GAPDH, an important enzyme of the intermediate cellular metabolism that, due to its catalytically active-critical Cys residue, has also been postulate to be a redox-sensor. OA-NO₂ is almost an order of magnitude more potent than the highly reactive oxidants in biology, hydrogen peroxide and peroxynitrite, towards GAPDH [13].

Covalent inhibitors display time-dependent inhibition and their potency has to be characterized by the analysis of the inactivation rate for different inhibitor concentrations. In the case of OA-NO₂, the determination of an inhibition constant for PknG is difficult for several reasons. The fact that OA-NO₂ concentration can not be readily increased over its critical micelle concentration and that increasing inhibitor concentration also increased the number of unspecific covalent modifications detected makes difficult the detailed kinetic analysis. In one embodiment of the present invention OA-NO₂ is administered to have a potent effect on kinase activity at micromolar concentrations of OA-NO₂ using PknG:OA-NO₂ ratios of 1:3 and 1:5.

The Cys-alkylated peptides showed an unexpected mass shift when detected using MALDI-TOF/TOF. In one embodiment of the present invention, laser ionization induces photodecomposition of cysteine-OA-NO₂ adducts generating the observed pattern with a mass shift of 198/200/202 Da. Decomposition of nitro-compounds during MALDI analysis was previously reported [37]. In contrast to cysteine modification, alkylated histidine showed the expected mass shift of 327 Da, demonstrating that the mass increment of 198/200/202 Da is a fingerprint of cysteine modification when detected by MALDI MS. This may explain why Cys-OA-NO₂ adducts were systematically not detected in MALDI experiments in previous work [13]. MALDI MS/MS spectra of the modified peptides with this atypical mass increments did not show fragments of the 198/200/202 Da modification, thus not allowing a structural characterization.

Thus, in accordance with the present invention, one embodiment exploits the unique structural characteristics of the multi-domain protein kinase PknG for the specific inhibition of its enzymatic activity. This embodiment represents a totally new mechanism for PknG inhibition involving the Rbx domain outside the catalytic domain. As contemplated in the present invention, electrophilic fatty-acids represent a new class of molecules for the specific inhibition of a small subset of kinases containing Rbx domain, and their use as PknG inhibitors.

### Working Examples

9-octadecenoic acid (oleic acid; OA) was purchased from Nu-Check Prep (Elysian, Minn.). 9- and 10-nitro-octadeca-9-cis-enoic acid (OA-NO₂) was prepared as previously [16]. GSH, DTT, bathophenanthrolinedisulfonic acid disodium salt (BPS), iodoacetamide (IAM) and 8-anilino-1-naphthalenesulfonic acid ammonium salt (ANS) were from Sigma. C18-Omix® Pipette tips for sample preparation were from Varian (Lake Forest, Calif.). Sequencing grade modified trypsin was from Promega (Madison, Wis.).

Full-length PknG was overexpressed in Escherichia coli BL21(DE3) cells grown for 24 h at 30° C. without IPTG and supplemented with 100 μM FeCl₃. PknG was purified as described before [5].

Site-directed mutagenesis of PknG was performed by PCR on pET-28a-74PknG using QuikChange II Site-Directed Mutagenesis Kit (Agilent Technologies). The sequence of the construct (PknG 74-406, Δ 74/TPR) was verified by DNA sequencing. PknG A74/TPR was overexpressed in E. coli BL21(DE3) cells grown at 30° C. until OD₆₀₀=0.6, then ON at 14° C. after addition of 1 mM IPTG. PknG Δ 74/TPR purification was performed as previously [5].

GarA (Rv1827) expression was performed in E. coli BL21(AI). Cells were grown in LB medium supplemented with 0.1% glucose and 10 μg/μL tetracycline at 37° C. until OD₆₀₀=0.6, then for 18 h at 22° C. after addition of 1 mM IPTG and 0.02% arabinose. The cells were harvested by centrifugation and re-suspended in buffer A (5 mM NaH₂PO₄, 50 mM Na₂HPO₄, 500 mM NaCl, 5% glycerol, 25 mM imidazole, pH 8.0) supplemented with Complete protease inhibitor cocktail (Roche)). GarA was first purified by metal-affinity chromatography on a HisTrap Ni²⁺-IMAC column (GE Healthcare) equilibrated in buffer A, using a linear imidazole gradient (20-400 mM). The GarA-containing fractions were dialyzed against buffer B (25 mM Tris-HCl, 150 mM NaCl, 5% glycerol, 1 mM DTT, pH 7.6), and the His₆ tag was removed by incubation for 24 h at 18° C. in the presence of His₆-tagged TEV endoprotease at a 1:30 ratio followed by separation on Ni-NTA agarose column (Qiagen). The untagged GarA was then further purified by size-exclusion chromatography on a Superdex 75 26/60 column (GE Healthcare) equilibrated in buffer B without DTT.

Protein phosphorylation reactions were performed in 50 mM HEPES buffer pH 7.0 containing 2 mM MnCl₂ and 100 μM ATP. Activity of PknG was assayed using recombinant GarA as substrate. The molar ratio of kinase:substrate ranged from 1:10 to 1:20. Reaction mixtures were incubated 30 min at 37° C. and substrate phosphorylation was evaluated by MALDI-TOF MS. The autophosphorylation activity of PknG was assessed by incubation of the enzyme in the presence of 2 mM MnCl₂ and 100 μM ATP for 40 min at 37° C. The samples were then digested with trypsin and phosphopeptides were detected by MS.

The effect of OA-NO₂—PknG pre-incubation times on kinase inhibition was assayed. PknG and OA-NO2 were incubated and at different time points aliquots of treated and control enzymes were removed for kinase activity determination. Activity assay was performed using Kinase Glo® Plus Luminiscent Kinase Assay (Promega) according to manufacturer guidelines. Briefly, activity was tested using GarA as substrate (kinase:substrate ratio was 1:25) and remaining ATP was quantified by luminescence after 30 min incubation time at 37° C. For each time point the inhibition relative to control enzyme is plotted.

Proteolytic digestion was carried out by incubating the proteins with trypsin in 70 mM ammonium bicarbonate, pH 8.0, for 12 h at 37° C. (enzyme-substrate ratio 1:10). Mass spectra of peptides mixtures were acquired in a 4800 MALDI TOF/TOF instrument (Applied Biosystems) in positive ion reflector or linear mode using a matrix solution of α-cyano-4-hydroxycinnamic acid in 0.2% trifluoroacetic acid in acetonitrile-H₂O (50%, v/v) and were externally calibrated using a mixture of standard peptides (Applied Biosystems). The molecular mass of the native and phosphorylated GarA was determined using a sinapinic acid matrix (10 mg/mL in acetonitrile-H₂O 50%, 0.2% trifluoroacetic acid). Alternatively, a linear ion trap mass spectrometer (LTQ Velos, Thermo) coupled on line with a nano-liquid chromatrography system (easy-nLC, Proxeon-Thermo) was used for peptide mixtures analysis. Peptides were separated on a reversed-phase column (EASY-column™ 100 mm, ID75 μm, 3 μm, C18-A2 from Proxeon) and eluted with a linear gradient of acetonitrile 0.1% formic acid (0-60% in 60 min) at a flow rate of 400 μL/min. Electrospray voltage was 1.40 kV and capillary temperature was 200° C. Peptides were detected in the positive ion mode using a mass range of 300-2000 in the data-dependent triple play MS2 mode (full scan followed by zoom scan and MS/MS of the top 5 peaks in each segment).

Native PknG (ranging from 5-10 μM) in 70 mM ammonium bicarbonate, pH 8.0, was incubated for 10 min at 25° C. with OA-NO₂ (0-100 μM) or IAM (0-500 μM) and kinase activity was immediately measured. As a control, PknG was exposed to OA-NO₂ vehicle (methanol) in the same conditions. In addition for OA-NO₂ experiments, kinase activity in the presence of equivalent concentration of OA was determined. In some experiments, after nitroalkene treatment PknG was incubated with DTT (42 mM) or GSH (24 mM) for 10 min at 25° C. and enzymatic activity was re-determined. The activity of control PknG in the presence and absence of the thiol containing reagents was also assayed. Previous to enzymatic digestions, excess of DTT was removed by immobilization of PknG on reverse-phase Poros 10 R2 beads (Applied Biosystems).

PknG tryptic peptides were isolated by reverse-phase HPLC (Vydac® C18; 2.1×100 mm) and fractions including cysteine-containing peptides were selected after mass analysis by MALDI-TOF. Selected fractions were dried, re-suspended in 70 mM ammonium bicarbonate, pH 8.0 and treated with OA-NO₂ (1:4) for 10 min at 25° C. Peptide modification was analyzed by MALDI MS and ESI MS.

The experiments using rabbit muscle GAPDH as model protein were carried out as previously reported [13].

Control and OA-NO₂-treated PknG were loaded onto OMIX C4 pipette tips (Agilent Technologies) and flow-through was collected. Non-bound fraction was incubated for 15 min at 25° C. with 40 mM DTT and iron was determined spectrophotometrically using BPS as previously described [17].

Concentration of ANS was determined using the molar extinction coefficient 8=5000 M⁻¹ cm⁻¹ at 350 nm [18]. PknG exposed to OA-NO₂ in the same experimental conditions stated above was diluted at 1 μM, dialyzed to remove excess OA-NO₂ and incubated with 16 μM ANS. As controls, PknG natively folded and thermally denatured were incubated with ANS.

Fluorescence was collected on Cary Eclipse fluorescence spectrophotometer (Varian, Inc.) with the excitation wavelength set on 350 nm and emission between 370-650 nm.

Circular dichroism (Far-UV) spectroscopy, was performed on OA-NO₂-treated and OA-treated PknG. CD spectra were recorded between 190 and 260 nm on an Aviv 215 spectropolarimeter (Aviv Biomedical), using a cylindrical cell with a 0.02 cm path length and an averaging time of 1s per step, with protein samples at 0.5 mg/mL in 25 mM Tris-HCl, 100 mM NaCl, glycerol 5% pH 8.0. Five consecutive scans from each sample were merged to produce an averaged spectrum and corrected using buffer baselines measured under the same conditions. Data were normalized to the molar peptide bond concentration and path length and expressed as Mean Residue Ellipticity ([0] degree·cm²·dmol⁻¹).

The bioinformatics procedures entailed searching protein sequence homologs to PknG at NCBI's NR database using CS-Blast [19], Psi-Blast [20] and HHsenser [21]. All searches were run performing both gapped and ungapped alignments, in order to selectively detect proteins carrying both Rbx and Ser/Thr protein kinase domains. Significant hits (Blast E-) values<1e⁻¹⁰) found with all methods with sequence coverage >75% were kept. Multiple sequence alignments (MSAs) were computed with Mafft [22], T-Coffee [23] and Prank [24]. Such MSAs were manually analyzed in order to detect sequences with N-terminal Rbx motifs (all had the kinase domain). Distance-based phylograms for a subset of 652 sequences (lengths between 500 and 800 aminoacids, pairwise identities <76%) were computed with BioNJ [25].For proteins with the Rbx domain, maximum-likelihood phylogenetic trees were built by way of PhyML [26].

PknG Inhibition by OA-NO2.

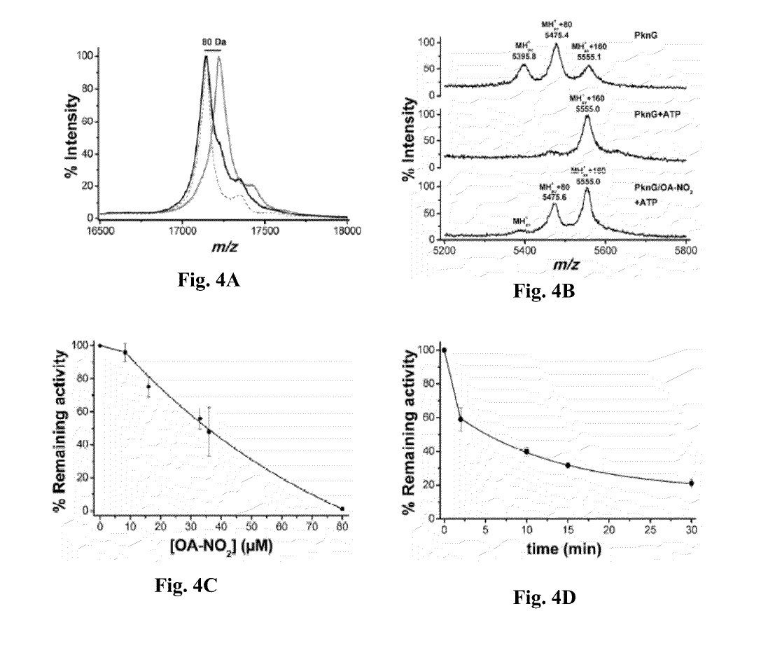

To evaluate whether the electrophilic nitro-oleic acid has an effect on PknG kinase activity, the enzyme was treated with different concentrations of OA-NO₂ (below its critical micelle concentration) and its remaining activity was measured using a recombinant protein substrate, GarA. Native and phosphorylated GarA were detected by MALDI-TOF MS as ions of m/z 17145 and m/z 17222 respectively, as before [27]. The mass shift corresponds to the incorporation of one phosphate group (80 Da) into the native sequence (FIG. 4A). Kinase activity of PknG exposed to micromolar concentrations of nitro-oleic acid (molar excess ranging from 1:1 to 1:3) was noticeably inhibited. The ability to phosphorylate GarA was clearly decreased after 10 minutes of preincubation of the kinase with OA-NO₂ (30 μM, molar ratio OA-NO₂: PknG 3:1) (FIG. 4A). As a control experiment PknG was exposed to equivalent concentrations of oleic acid or methanol (vehicle) and no effect on kinase activity was observed (data not shown).

The effect of OA-NO₂ treatment on PknG autophosphorylation was also evaluated to confirm that the inhibition observed reflects a general loss of the kinase activity more than a substrate specific effect (FIG. 4B). PknG is able to autophosporylate its N-terminal sequence at specific Thr residues [5]. The spectrum obtained in the linear mode after tryptic digestion of native PknG showed the presence of unphosphorylated (m/z 5395.8), monophosphorylated (m/z 5475.4) and diphosphorylated (m/z 5555.1) ions, as previously (FIG. 4B) [5]. When native PknG was incubated with ATP under phosphorylation conditions and then digested, the most intense ion observed corresponded to the diphosphorylated specie while unphosphorylated and monophosphorylated ions were almost undetectable (FIG. 4B). On the other hand, OA-NO₂ (30 μM) impairs the conversion of the un- and monophosphorylated forms of the kinase into the fully phosphorylated species, indicating that the autocatalytic activity is also inhibited by the nitrated fatty acid (FIG. 4B).

The effect of OA-NO₂ on kinase activity was tested using inhibitor concentration ranging from 0 to 80 μM. As shown in FIG. 4C there was a dose-dependent inactivation of PknG upon pre-incubation of the enzyme with different OA-NO₂ concentrations for 10 min. Under these experimental conditions 50% of enzyme inhibition was reached with 35 μM of OA-NO₂.

To further characterize the effect of the nitrated fatty acid on PknG, we studied the time course of enzyme inhibition. PknG and OA-NO₂ (or vehicle as control) was pre-incubated for time periods ranging from 0 to 30 min, and kinase activity was measured using a commercial luminescent kinase assay (Kinase Glo®)). Under these experimental conditions the control activity varies less than 10% during the 30 min incubation time. The percentage of remaining kinase activity was calculated with respect to control activity for the same time point. As shown in FIG. 4D the exposure of PknG to OA-NO₂ decreased kinase activity in a time-dependent manner. The dependence of PknG inhibition on enzyme-OA-NO₂ reaction times suggests that the effect of OA-NO₂ is mediated by a covalent modification of PknG.

Cysteines at the Rbx domain are the main target of OA-NO₂.

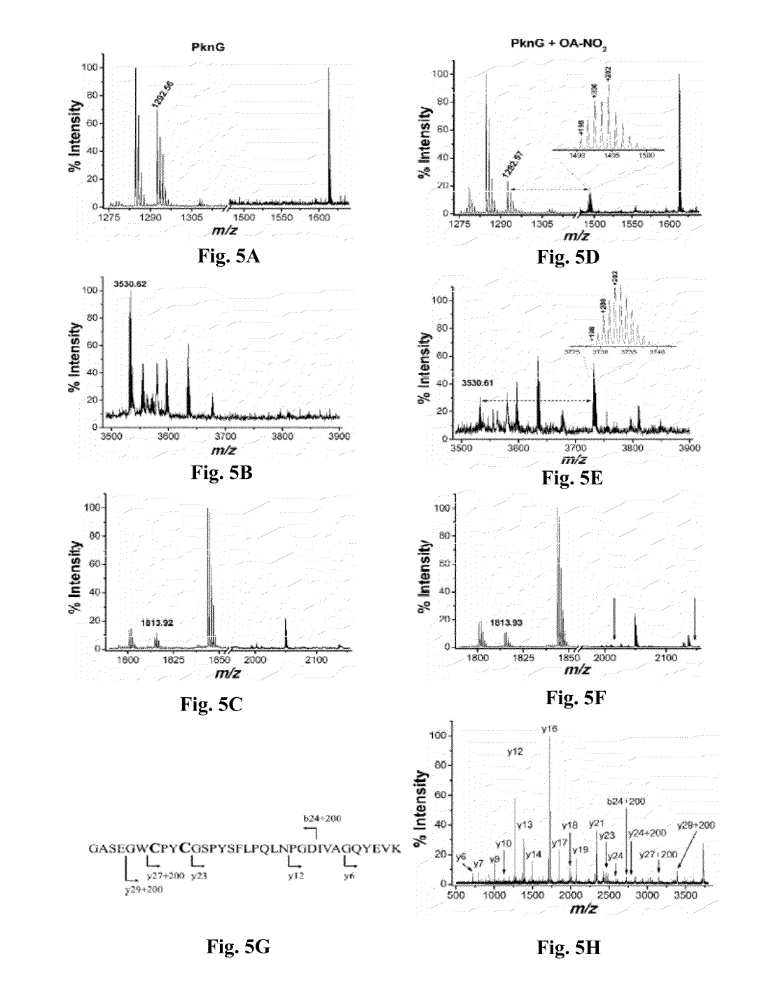

In order to identify the PknG residues that may account for the inhibition of the kinase, samples were digested with trypsin and peptide mixtures were analyzed by MS (FIGS. 5A-5H). MALDI-TOF mass spectra of OA-NO₂-treated samples (OA-NO₂ concentration up to 3504) showed a clear consumption of only two peptides; each one containing a different CXXCG (Seq. No. 12) motif of the Rbx domain: m/z 1292.56 and 3530.62 (considering the intra-molecular disulfide bond) (FIGS. 5A-5F). No other peptide showed a significant change in signal intensity compared with control spectra (data not shown). The expected mass shift for the incorporation of a molecule of OA-NO₂ is 327 Da [13]. However, under this experimental condition we were unable to detect any peptide with this mass increment. A detailed analysis of MALDI-TOF/TOF mass spectra of OA-NO₂ treated samples showed the systematic appearance of a cluster of peaks with 198, 200 and 202 Da higher mass than the CXXCG (Seq. No. 12)-containing peptides (FIGS. 5D and 5E). MS/MS spectra of the observed ions (m/z 1492.54 and 3730.59) confirmed that they corresponded to modified peptides containing the sequences Seq. No. 1 and Seq. No. 3, respectively. FIG. 5G shows the MS/MS spectra of the precursor ion corresponding to modified sequence Seq. No. 3 (GASEGWC₁₂₈PYC₁₃₁GSPYSFLPQLNPGDIVAGQYEVK) with a mass increment of 200 Da. The presence of native y-ion series up to y23 together with the detection of the modified y27 (+200 Da), indicate that any of the residues in between (i.e. ₁₂₈CPYC₁₃₁) might be modified by OA-NO₂ (FIG. 5G). Based on the presence of small signals corresponding to unmodified y24, y24+200 Da, unmodified y26 and y26+200 Da we can postulate that ion m/z=3730.59 is actually a mixture of singly modified peptides where C₁₂₈ or C₁₃₁ have been individually modified by OA-NO₂ (Table 1 shown below). The observed mass shift after OA-NO₂ treatment was 198/200 Da.

TABLE 1
MS/MA data of OA-NO₂ modified peptide Sequence ID No. 3.
List of theoretical m/z values of fragment ions from peptide
Seq. No. 003 and its coresponding ion +200 Da. y- and
b- ions detected by MALDI MS are highlighted in bold.

| # | b | b + 200 | Residue | y | y + 200 | # |
|---|---|---|---|---|---|---|
| 1 | 58.03 | 258.03 | G | | | 33 |
| 2 | 129.07 | 329.07 | A | 3475.60 | 3675.60 | 32 |
| 3 | 216.10 | 416.10 | S | 3404.56 | 3604.56 | 31 |
| 4 | 345.14 | 545.14 | E | 3317.53 | 3517.53 | 30 |
| 5 | 402.16 | 602.16 | G | 3188.49 | 3388.49 | 29 |
| 6 | 588.24 | 788.24 | W | 3131.47 | 3331.47 | 28 |
| 7 | 690.24 | 890.24 | C | 2945.99 | 3145.39 | 27 |
| 8 | 787.30 | 987.30 | P | 2842.38 | 3042.38 | 26 |
| 9 | 950.36 | 1150.36 | Y | 2745.32 | 2945.32 | 25 |
| 10 | 1052.36 | 1252.36 | C | 2582.26 | 2782.26 | 24 |
| 11 | 1109.38 | 1309.38 | G | 2479.25 | 2679.25 | 23 |
| 12 | 1196.41 | 1396.41 | S | 2422.23 | 2622.23 | 22 |
| 13 | 1293.47 | 1493.47 | P | 2335.20 | 2535.20 | 21 |
| 14 | 1456.53 | 1656.53 | Y | 2238.15 | 2438.15 | 20 |
| 15 | 1543.56 | 1743.56 | S | 2075.08 | 2275.08 | 19 |
| 16 | 1690.63 | 1890.63 | F | 1988.05 | 2188.05 | 18 |
| 17 | 1803.72 | 2003.72 | L | 1840.98 | 2040.98 | 17 |
| 18 | 1900.77 | 2100.77 | P | 1727.90 | 1927.90 | 16 |
| 19 | 2028.83 | 2228.83 | Q | 1630.84 | 1830.84 | 15 |
| 20 | 2141.91 | 2341.91 | L | 1502.79 | 1702.79 | 14 |
| 21 | 2255.95 | 2455.95 | N | 1389.70 | 1589.70 | 13 |
| 22 | 2353.01 | 2553.01 | P | 1275.66 | 1475.66 | 12 |
| 23 | 2410.03 | 2610.03 | G | 1178.61 | 1378.61 | 11 |
| 24 | 2525.05 | 2725.05 | D | 1121.58 | 1321.58 | 10 |
| 25 | 2638.14 | 2838.14 | I | 1006.56 | 1206.56 | 9 |
| 26 | 2737.21 | 2937.21 | V | 893.47 | 1093.47 | 8 |
| 27 | 2808.24 | 3008.24 | A | 794.41 | 994.41 | 7 |
| 28 | 2865.27 | 3065.27 | G | 723.37 | 923.37 | 6 |
| 29 | 2993.32 | 3193.32 | Q | 666.35 | 866.35 | 5 |
| 30 | 3156.39 | 3356.39 | Y | 538.29 | 738.29 | 4 |
| 31 | 3285.43 | 3485.43 | E | 375.22 | 575.22 | 3 |
| 32 | 3384.50 | 3584.50 | V | 246.18 | 446.18 | 2 |
| 33 | 3512.59 | 3712.59 | K | 147.11 | 347.11 | 1 |

Similarly, MS/MS analysis of the other modified Rbx peptide (m/z 1492.54) showed that only daughter ions that contain the CWNC (Seq. No. 14) residues appeared with a modified mass (data not shown). Although enough sequence information to identify the modified residue(s) within this motif was not available, based on the previously reported reactivity of the nitrated fatty acid towards nucleophilic residues, these results strongly suggest that the Cys residues of those peptides are the main target of OA-NO₂ [13].

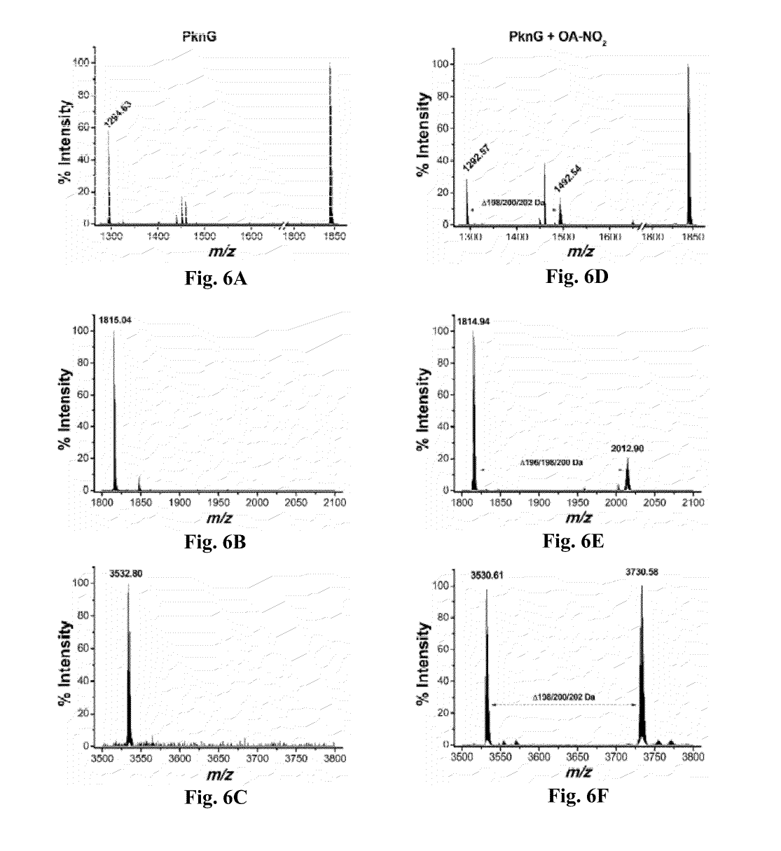

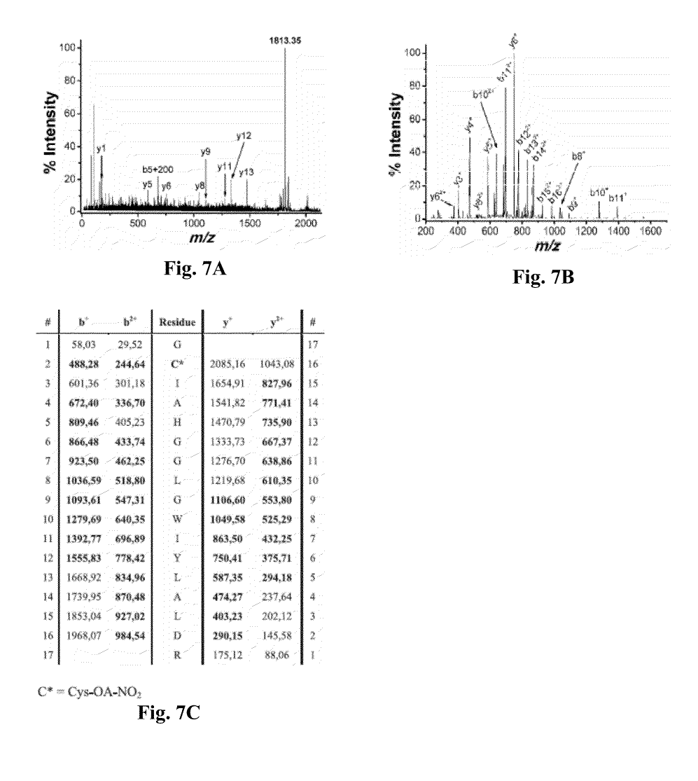

To further characterize this modification and the observed atypical mass shift, all three PknG native tryptic peptides that contain Cys residues (m/z 1292.57, m/z 1813.92 and m/z 3530.60) were isolated by RP-HPLC and then treated with OA-NO₂. The peptide with the sequence Seq. No. 13 (m/z 1813.92) is the only tryptic peptide of PknG that contains a single Cys. The modification pattern previously observed was found for all those three Cys-containing peptides treated with OA-NO₂ (FIG. 6A-6F). The mass shift for the peptide containing a single Cys was 196/198/200 Da. This observation is in fully agreement with the observed mass shift of 198/200/202 Da for the CXXCG (Seq. No. 12) containing peptide with respect to the native peptide with an intra-molecular disulfide bond. These results indicate that the presence of a single Cys residue is enough to generate the observed mass shift in MALDI-TOF MS experiments. In contrast, the analysis of those same peptides by electrospray ionization-MS showed the expected mass increment of 327 Da, but not the 198/200/202 Da pattern (FIGS. 7A and 7B). MS/MS spectra of the modified peptides allowed us to unequivocally identify Cys as the modified residue by OA-NO₂ (FIGS. 7B and 7C). Altogether, these results support the hypothesis of MALDI ionization induced decomposition of Cys-nitrated fatty acid adducts. However, MALDI MS/MS spectra did not provide enough information to interpret the structure of the modification responsible of the 198/200/202 Da mass shift.

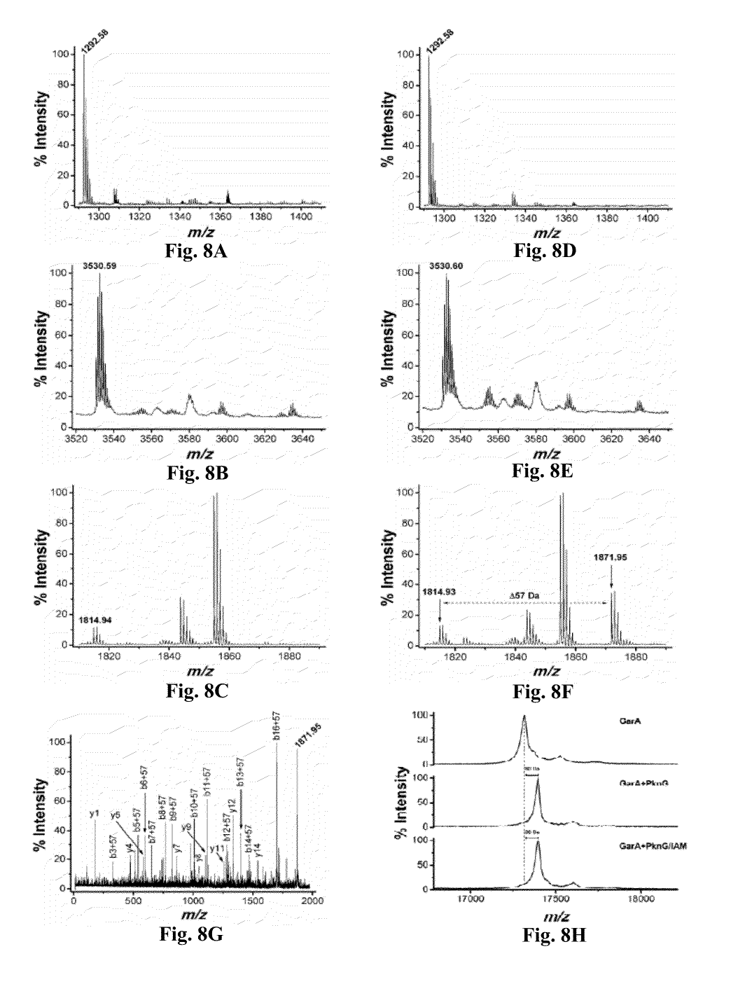

PknG sequence Seq. No. 13 contains a unique non-rubredoxin Cys (C₁₅₆). Noticeably, no consumption of the peptide containing this free Cys was observed upon treatment of PknG with OA-NO₂ (FIGS. 5C and 5F). In full agreement with this observation, no appearance of a new signal with the previously observed mass shift (198/200/202 Da) or the theoretical expected mass shift (+327 Da) was observed. To gain further insights into the reactivity of PknG-Cys residues, we performed experiments with iodoacetamide, as a model reagent for cysteine alkylation. No modification in Rbx cysteines was observed for IAM concentrations up to 150 μM (FIGS. 8A-8H). Conversely, the unbound Cys₁₅₆ was alkylated by IAM under equal experimental settings, pointing to a very different reactivity of OA-NO₂ vs. other alkylating reagents toward Cys residues in PknG. While treating PknG with the nitrated fatty acid modified cysteines tightly bound to the metal ion and inhibited the enzyme, treatments with IAM only resulted in modification of the free Cys without affecting kinase activity (FIGS. 8A-8H). Therefore, at the described experimental conditions, FIGS. 8A-8H shows for the first time that Rbx-Cys, typically reported as non-reactive toward the classical alkylating reagents, are the main targets of the reaction with the electrophilic nitroalkene.

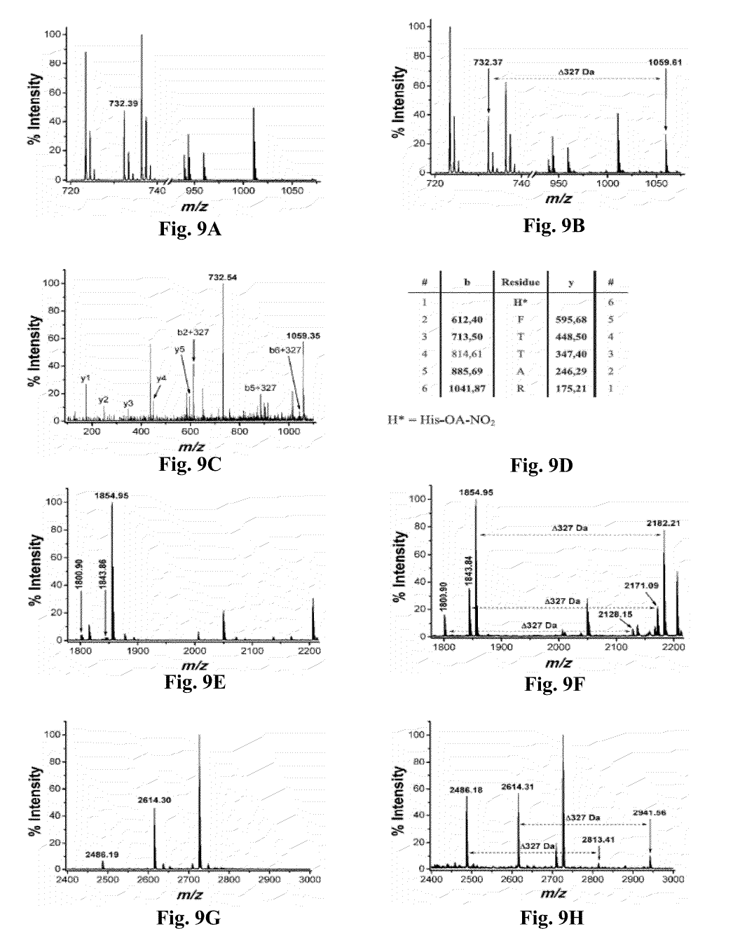

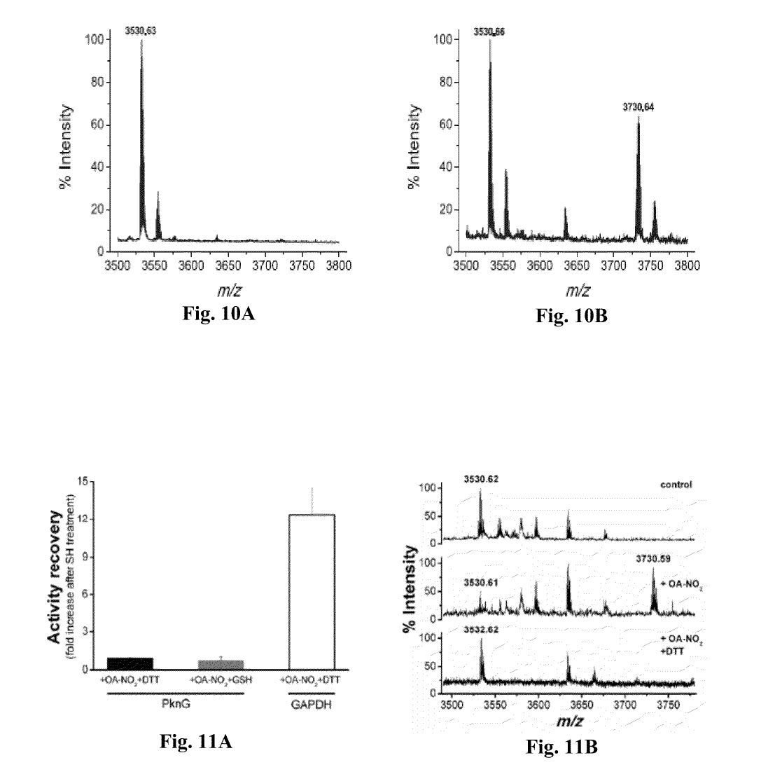

In experiments performed with higher concentrations of OA-NO₂ (50-80 μM; molar ratio PknG:OA-NO₂ from 1:5 to 1:10) further showed the modification of several His residues in a very low yield. In agreement with this observation, no significant consumption of His-containing peptides was detected (FIGS. 9A-9H and Table 2 shown below).

TABLE 2
Identification of OA-NO₂-modified residues in PknG

| | Peptide | Modified | | OA-NO₂:PknG ratio^{#} | |
|---|---|---|---|---|---|
| SEQ ID No. | from-to | residue | Assigned sequence^{a} (observed m/z) | low | high |

Peptides containing modified cysteine residues (Δm = 198/200/202 Da)

| | | | | | |
|---|---|---|---|---|---|
| Seq. No. 1 | 105-111 | C₁₀₆ or C₁₀₉ | FCWNCGR | x | x |
| | | | (1081.31/1083.30/1085.33) | | |
| Seq. No. 2 | 105-115 | C₁₀₆ or C₁₀₉ | FCWNCGRPVGR | x | x |
| | | | (1490.51/1492.54/1496.55) | | |
| Seq. No. 3 | 122-154 | C₁₂₈ or C₁₃₁ | GASEGWCPYCGSPYSFLPQLNPGDIVAGQYEVK | x | x |
| | | | (3728.55/3730.59/3732.60) | | |

Peptides containing modified histidine residues (Δm = 327 Da)

| | | | | |
|---|---|---|---|---|
| Seq. No. 4 | 182-199 | H₁₈₅ | GLVHSGDAEAQAMAMER | x |
| | | | (2171.09) | |
| Seq. No. 5 | 420-443 | H₄₃₀ or H₄₄₀ | STFGVDLLVAHTDVYLDGQVHAEK | x |
| | | | (2941.56) | |
| Seq. No. 6 | 488-510 | H₄₈₈ | HGALDADGVDFSESVELPLMEVR | x |
| | | | (2813.41) | |
| Seq. No. 7 | 558-573 | H₅₅₈ | HFTEVLDTFPGELAPK | x |
| | | | (2128.15) | |
| Seq. No. 8 | 635-640 | H₆₃₅ | HFTTAR | x |
| | | | (1059.61) | |
| Seq. No. 9 | 698-714 | H₇₀₂ or H₇₁₁ | ASTNHILGFPFTSHGLR | x |
| | | | (2182.21) | |
| Seq. No. 10 | 733-743 | H₇₃₃ | HRYTLVDMANK | x |
| | | | (1674.91) | |

^{a}The sequence and alkylation sites were confirmed by MS/MS analysis.
^{#}Low OA-NO₂:PknG ratio means a molar ratio in between 1:1 and 3:1 ([OANO₂] between 8 μM and 30 μM). High OA-NO₂:PknG ratio means a molar ratio 10:1 ([OANO₂] = 80 μM)

In contrast to cysteine modification, alkylated histidine showed the expected mass shift of 327 Da when analyzed by MALDI MS and ESI MS. MS/MS analysis of these peptides allowed us to confirm that the incorporation of the nitrated fatty acid took place at a His residue (Table 2 and FIGS. 9A-9H). To finally confirm that inhibition is a consequence of Cys but not His modification, we repeated the experiments using an active PknG construction where N-terminal and TPR domain were removed (PknGΔ74/TPR). This truncated construction preserves only His185 out of the seven previously modified His residues. After the exposure of PknGΔ74/TPR to OA-NO₂ the enzyme is fully inhibited, the Cys-containing peptides from the Rbx domain appeared alkylated and we observed another set of His residues with low yield of modification (Table 3 shown below and FIGS. 10A-10B).

TABLE 3
Identification of modified residues within pknG Δ74/TPR sequence.

| | | | |
|---|---|---|---|
| Seq. ID No. | Peptide from-to | Modified residue | Assigned sequence^{a} |

Peptides containing modified cysteine residues (Δm = 198/200/202 Da)

| | | | |
|---|---|---|---|
| Seq. No. 1 | 105-111 | C₁₀₆ or C₁₀₉ | FCWNCGR |
| Seq. No. 2 | 105-115 | C₁₀₆ or C₁₀₉ | FCWNCGRPVGR |
| Seq. No. 3 | 122-154 | C₁₂₈ or C₁₃₁ | GASEGWCPYCGSPYSFLPQLNPGDIVAGQYEVK |

Peptides containing modified histidine residues (Δm = 327 Da)

| | | | |
|---|---|---|---|
| Seq. No. 4 | 182-199 | H₁₈₅ | GLVHSGDAEAQAMAMAER |
| Seq. No. 11 | 200-222 | H₂₀₇ or H₂₁₉ | QFLAEVVHPSIVQIFNFVEHTDR |

Using both enzyme constructions, Cys-Rbx are the only modified residues detected using the OA-NO₂ concentrations up to 35 μM, that are sufficient to render a noticeable loss of PknG activity. Altogether these data indicate that Cys residues at the Rbx domain are the main targets of OA-NO₂ and may account for the observed enzyme inhibition.

Irreversible PknG Inhibition by the Reversible OA-NO₂ Mediated Nitroalkylation of the Kinase.

We have previously reported that nitrated fatty acids are capable to inhibit GAPDH activity by modification of nucleophilic amino acid residues in a thiol-reversible manner [13]. Herein, we analyze the reversibility of PknG nitroalkylation. Treatment of nitroalkylated PknG with DTT (42 mM) or GSH (24 mM) was unable to recover the kinase activity (FIG. 11A). Under the same experimental conditions GAPDH activity, used as a control, was almost fully recovered (FIG. 11A). PknG control activity was measured in the presence of the thiol-containing reagents. Neither DTT nor GSH has an effect per se on kinase activity at these experimental conditions.

To evaluate the reversibility of the chemical modification of His and Cys residues in PknG, OA-NO₂ treated samples were further exposed to thiol-containing reagents (DTT or GSH) before protein digestion and MS analysis. We initially analyzed the peptide containing two Rbx-Cys (Seq. No. 3). The spectrum clearly showed that nitroalkylation of this peptide in PknG is reversible as we were unable to detect its modified form after exposure to DTT. In addition, native cysteine-containing peptides were fully recovered by treatment with DTT (FIG. 11B). Moreover, we were unable to detect any Cys- or His-nitrated fatty acid adducts after the treatment with DTT or GSH (data not shown). Although nitroalkylation turns to be reversible (as expected), the effect of OA-NO₂ on PknG activity was irreversible (FIG. 11A). Altogether, these results indicate that OA-NO₂ is inactivating PknG by an unusual mechanism involving a more permanent change in PknG structure or function that persist even when the chemical modification is already removed.

OA-NO₂ Induces Iron Release from Rbx Domain.

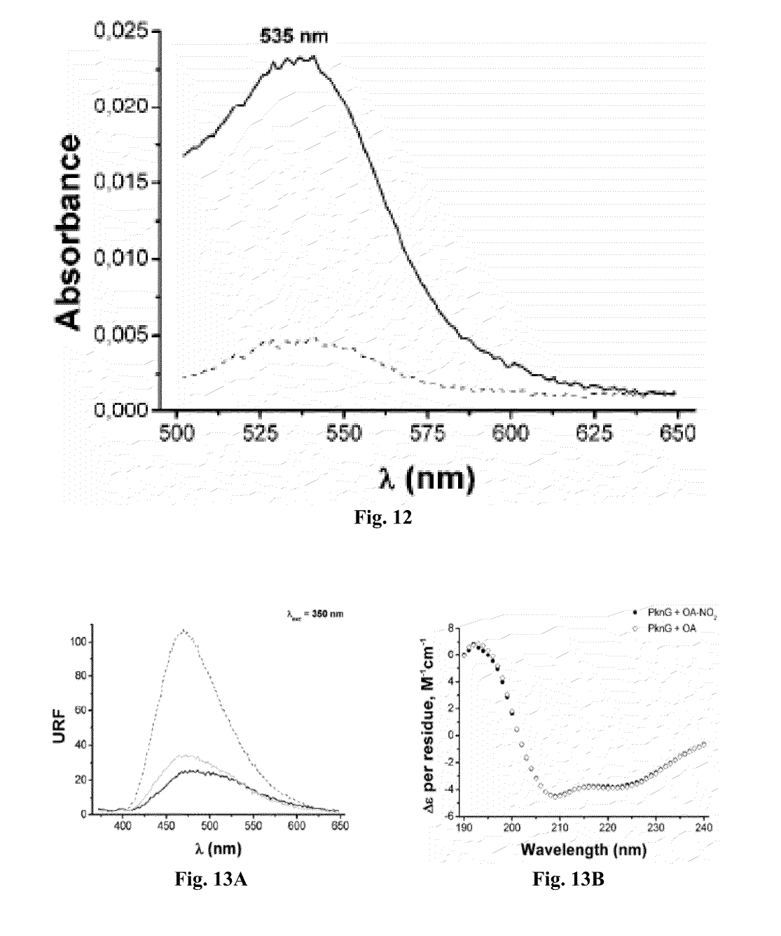

The rubredoxin domain contains an iron ion which is coordinated by the sulphurs of four conserved cysteine residues forming an almost regular tetrahedron. Fe³⁺ is hardly removed from Rbxs [28, 29]. To evaluate the effect of Cys nitroalkylation on the metal center of the Rbx domain, we measured the amount of iron released upon OA-NO₂ treatment, using a specific ligand for its spectrophotometric determination. Non protein-bound iron present in control and OA-NO₂ treated PknG samples was recovered, reduced with DTT and quantified spectrophotometrically. The results clearly showed that PknG nitroalkylation leaded to iron release from the Rbx domain (FIG. 12). Our data showed that 70% of the iron present at the Rbx domain was released after the protein exposure to 50 μM of OA-NO₂. As a positive control total iron in native protein was determined after Rbx domain disruption by protein digestion (data not shown).

PknG Inhibition by OA-NO₂ is not the Consequence of a Global Change in PknG Structure.

In order to address if the effect of OA-NO₂ could be mediated by an unspecific global distortion of protein tridimensional structure as a consequence of the introduction of a quite large hydrophobic molecule, we analyzed global changes in protein structure by different approaches. We incubated OA-NO₂-treated enzyme with ANS, a fluorescent probe that binds to hydrophobic patches on proteins with a concomitant change in emission spectrum (FIG. 13A). The same spectra were obtained with native and OA-NO₂-modified PknG, showing that the overall hydrophobic surface of the protein was not affected by the modification (FIG. 13A). These results suggest that the observed effect on enzyme activity was not due to a large protein structural change. In order to discard minor changes in secondary structure we performed circular dichroism experiments on PknG previously treated with OA-NO₂ or OA (FIG. 13B). The comparison of the spectra showed no difference between both samples. Overall, the results suggest that PknG inhibition by OA-NO₂ can not be attributed to a general structural modification as a result of Cys nitroalkylation and iron release from the Rbx domain.

Rbx and Kinase Domain Co-Occurrence is Restricted to Few Actinomycetales.

The specificity of OA-NO₂ reactivity towards Cys residues in the Rbx domain of PknG raised the possibility of a selective inhibition of certain kinases containing this domain. Employing bioinformatics, we analyzed the co-ocurrence of Rbx and kinase domains.

Multiple sequence alignments of PknG orthologs showed a minority of PknG-like kinases harboring the CXXCG (Seq. No. 12) motif linked to Rbx domains (FIG. 1). In a subset of 652 PknG-like kinases the Rbx domain was present in less than 10% of them (FIG. 1). The same trend was observed in more thorough datasets, e.g.: from 1294 proteins, just 88 had the N-terminal motif. The co-ocurrence of Rbx and kinase domains in different species was further analyzed. FIG. 1B displays a maximum-likelihood phylogenetic tree for a set of 52 sequences with sequence identities below 90% and above 30%. At sequence identity levels above 25%, all but two PknG homologs carrying both domains were confined to few suborders of the Actinomycetales. The two exceptions were Acetivibrio cellulolyticus (Firmicutes) and Ktedonobacter racemifer (Chloroflexi).

Conservation of the catalytic domain, as well as variability of the N-terminus, has previously been described for a number of PknGs [30, 31]. However, presence/absence of the Rbx domain and sequence conservation levels had not been analyzed, to our knowledge. Interestingly, joint occurrence of kinase and Rbx domains is restricted to bacteria from the Actynomycetales order, including pathogenic and non-pathogenic mycobacteria. It is worth noting that the sequences indicated by a dot in FIG. 1A correspond to different species of Corynebaacterium lacking the CXXCG (Seq. No. 12) motifs of the Rbx domain.

### REFERENCES

[1] Wehenkel, A.; Bellinzoni, M.; Grana, M.; Duran, R.; Villarino, A.; Fernandez, P.; Andre-Leroux, G.; England, P.; Takiff, H.; Cervenansky, C.; Cole, S. T.; Alzari, P. M. Mycobacterial Ser/Thr protein kinases and phosphatases: physiological roles and therapeutic potential. Biochimica et biophysica acta 1784:193-202; 2008.
[2] Szekely, R.; Waczek, F.; Szabadkai, I.; Nemeth, G.; Hegymegi-Barakonyi, B.; Eros, D.; Szokol, B.; Pato, J.; Hafenbradl, D.; Satchell, J.; Saint-Joanis, B.; Cole, S. T.; Orfi, L.; Klebl, B. M.; Keri, G. A novel drug discovery concept for tuberculosis: inhibition of bacterial and host cell signalling. Immunology letters 116:225-231; 2008.
[3] Cole, S. T.; Brosch, R.; Parkhill, J.; Gamier, T.; Churcher, C.; Harris, D.; Gordon, S. V.; Eiglmeier, K.; Gas, S.; Barry, C. E., 3rd; Tekaia, F.; Badcock, K.; Basham, D.; Brown, D.; Chillingworth, T.; Connor, R.; Davies, R.; Devlin, K.; Feltwell, T.; Gentles, S.; Hamlin, N.; Holroyd, S.; Hornsby, T.; Jagels, K.; Krogh, A.; McLean, J.; Moule, S.; Murphy, L.; Oliver, K.; Osborne, J.; Quail, M. A.; Rajandream, M. A.; Rogers, J.; Rutter, S.; Seeger, K.; Skelton, J.; Squares, R.; Squares, S.; Sulston, J. E.; Taylor, K.; Whitehead, S.; Barrell, B. G. Deciphering the biology of Mycobacterium tuberculosis from the complete genome sequence. Nature 393:537-544; 1998.
[4] Walburger, A.; Koul, A.; Ferrari, G.; Nguyen, L.; Prescianotto-Baschong, C.; Huygen, K.; Klebl, B.; Thompson, C.; Bacher, G.; Pieters, J. Protein kinase G from pathogenic mycobacteria promotes survival within macrophages. Science (New York, N.Y. 304:1800-1804; 2004.
[5] O'Hare, H. M.; Duran, R.; Cervenansky, C.; Bellinzoni, M.; Wehenkel, A. M.; Pritsch, O.; Obal, G.; Baumgartner, J.; Vialaret, J.; Johnsson, K.; Alzari, P. M. Regulation of glutamate metabolism by protein kinases in mycobacteria. Molecular microbiology 70:1408-1423; 2008.
[6] Cowley, S.; Ko, M.; Pick, N.; Chow, R.; Downing, K. J.; Gordhan, B. G.; Betts, J. C.; Mizrahi, V.; Smith, D. A.; Stokes, R. W.; Av-Gay, Y. The Mycobacterium tuberculosis protein serine/threonine kinase PknG is linked to cellular glutamate/glutamine levels and is important for growth in vivo. Molecular microbiology 52:1691-1702; 2004.
[7] Niebisch, A.; Kabus, A.; Schultz, C.; Weil, B.; Bott, M. Corynebacterial protein kinase G controls 2-oxoglutarate dehydrogenase activity via the phosphorylation status of the OdhI protein. The Journal of biological chemistry 281:12300-12307; 2006.
[8] Scherr, N.; Honnappa, S.; Kunz, G.; Mueller, P.; Jayachandran, R.; Winkler, F.; Pieters, J.; Steinmetz, M. O. Structural basis for the specific inhibition of protein kinase G, a virulence factor of Mycobacterium tuberculosis. Proceedings of the National Academy of Sciences of the United States of America 104:12151-12156; 2007.
[9] Sieker, L. C.; Stenkamp, R. E.; LeGall, J. Rubredoxin in crystalline state. Methods Enzymol 243:203-216; 1994.
[10] van Beilen, J. B.; Neuenschwander, M.; Smits, T. H.; Roth, C.; Balada, S. B.; Witholt, B. Rubredoxins involved in alkane oxidation. Journal of bacteriology 184:1722-1732; 2002.
[11] Tiwari, D.; Singh, R. K.; Goswami, K.; Verma, S. K.; Prakash, B.; Nandicoori, V. K. Key residues in Mycobacterium tuberculosis protein kinase G play a role in regulating kinase activity and survival in the host. The Journal of biological chemistry 284:27467-27479; 2009.
[12] Schopfer, F. J.; Batthyany, C.; Baker, P. R.; Bonacci, G.; Cole, M. P.; Rudolph, V.; Groeger, A. L.; Rudolph, T. K.; Nadtochiy, S.; Brookes, P. S.; Freeman, B. A. Detection and quantification of protein adduction by electrophilic fatty acids: mitochondrial generation of fatty acid nitroalkene derivatives. Free radical biology & medicine 46:1250-1259; 2009.
[13] Batthyany, C.; Schopfer, F. J.; Baker, P. R.; Duran, R.; Baker, L. M.; Huang, Y.; Cervenansky, C.; Branchaud, B. P.; Freeman, B. A. Reversible post-translational modification of proteins by nitrated fatty acids in vivo. The Journal of biological chemistry 281:20450-20463; 2006.
[14] Schopfer, F. J.; Cole, M. P.; Groeger, A. L.; Chen, C. S.; Khoo, N. K.; Woodcock, S. R.; Golin-Bisello, F.; Motanya, U. N.; Li, Y.; Zhang, J.; Garcia-Barrio, M. T.; Rudolph, T. K.; Rudolph, V.; Bonacci, G.; Baker, P. R.; Xu, H. E.; Batthyany, C. I.; Chen, Y. E.; Hallis, T. M.; Freeman, B. A. Covalent peroxisome proliferator-activated receptor gamma adduction by nitro-fatty acids: selective ligand activity and anti-diabetic signaling actions. The Journal of biological chemistry 285:12321-12333; 2010.
[15] Kelley, E. E.; Batthyany, C. I.; Hundley, N. J.; Woodcock, S. R.; Bonacci, G.; Del Rio, J. M.; Schopfer, F. J.; Lancaster, J. R., Jr.; Freeman, B. A.; Tarpey, M. M. Nitro-oleic acid, a novel and irreversible inhibitor of xanthine oxidoreductase. The Journal of biological chemistry 283:36176-36184; 2008.
[16] Baker, P. R.; Lin, Y.; Schopfer, F. J.; Woodcock, S. R.; Groeger, A. L.; Batthyany, C.; Sweeney, S.; Long, M. H.; Iles, K. E.; Baker, L. M.; Branchaud, B. P.; Chen, Y. E.; Freeman, B. A. Fatty acid transduction of nitric oxide signaling: multiple nitrated unsaturated fatty acid derivatives exist in human blood and urine and serve as endogenous peroxisome proliferator-activated receptor ligands. The Journal of biological chemistry 280:42464-42475; 2005.
[17] Blair, D.; Diehl, H. Bathophenanthrolinedisulphonic acid and bathocuproinedisulphonic acid, water soluble reagents for iron and cooper. Talanta 7:163-174; 1961.
[18] Weber, G.; Young, L. B. Fragmentation of Bovine Serum Albumin by Pepsin. I. the Origin of the Acid Expansion of the Albumin Molecule. The Journal of biological chemistry 239:1415-1423; 1964.
[19] Biegert, A.; Soding, J. Sequence context-specific profiles for homology searching. Proceedings of the National Academy of Sciences of the United States of America 106:3770-3775; 2009.
[20] Altschul, S. F.; Madden, T. L.; Schaffer, A. A.; Zhang, J.; Zhang, Z.; Miller, W.; Lipman, D. J. Gapped BLAST and PSI-BLAST: a new generation of protein database search programs. Nucleic acids research 25:3389-3402; 1997.
[21] Soding, J.; Remmert, M.; Biegert, A.; Lupas, A. N. HHsenser: exhaustive transitive profile search using HMM-HMM comparison. Nucleic acids research 34:W374-378; 2006.
[22] Katoh, K.; Toh, H. Recent developments in the MAFFT multiple sequence alignment program. Briefings in bioinformatics 9:286-298; 2008.
[23] Notredame, C.; Higgins, D. G.; Heringa, J. T-Coffee: A novel method for fast and accurate multiple sequence alignment. Journal of molecular biology 302:205-217; 2000.
[24] Loytynoja, A.; Goldman, N. An algorithm for progressive multiple alignment of sequences with insertions. Proceedings of the National Academy of Sciences of the United States of America 102:10557-10562; 2005.
[25] Guindon, S.; Gascuel, O. A simple, fast, and accurate algorithm to estimate large phylogenies by maximum likelihood. Systematic biology 52:696-704; 2003.
[26] Guindon, S.; Delsuc, F.; Dufayard, J. F.; Gascuel, 0. Estimating maximum likelihood phylogenies with PhyML. Methods in molecular biology (Clifton, N.J) 537:113-137; 2009.
[27] Villarino, A.; Duran, R.; Wehenkel, A.; Fernandez, P.; England, P.; Brodin, P.; Cole, S.
T.; Zimny-Arndt, U.; Jungblut, P. R.; Cervenansky, C.; Alzari, P. M. Proteomic identification of M. tuberculosis protein kinase substrates: PknB recruits GarA, a FHA domain-containing protein, through activation loop-mediated interactions. Journal of molecular biology 350:953-963; 2005.
[28] Bonomi, F.; Iametti, S.; Kurtz, D. M., Jr.; Ragg, E. M.; Richie, K. A. Direct metal ion substitution at the [M(SCys)4]2P site of rubredoxin. J Biol Inorg Chem 3:595-505; 1998.
[29] Messerschmidt, A.; Huber, R.; Poulos, T.; Wieghardt, K., eds. Handbook of metalloproteins. Hoboken, N.J.: John Wiley & Sons, Inc.; 2001.
[30] Fiuza, M.; Canova, M. J.; Zanella-Cleon, I.; Becchi, M.; Cozzone, A. J.; Mateos, L. M.; Kremer, L.; Gil, J. A.; Molle, V. From the characterization of the four serine/threonine protein kinases (PknA/B/G/L) of Corynebacterium glutamicum toward the role of PknA and PknB in cell division. The Journal of biological chemistry 283:18099-18112; 2008.
[31] Narayan, A.; Sachdeva, P.; Sharma, K.; Saini, A. K.; Tyagi, A. K.; Singh, Y. Serine threonine protein kinases of mycobacterial genus: phylogeny to function. Physiological genomics 29:66-75; 2007.
[32] Alber, T. Signaling mechanisms of the Mycobacterium tuberculosis receptor Ser/Thr protein kinases. Curr Opin Struct Biol 19:650-657; 2009.
[33] Nott, T. J.; Kelly, G.; Stach, L.; Li, J.; Westcott, S.; Patel, D.; Hunt, D. M.; Howell, S.; Buxton, R. S.; O'Hare, H. M.; Smerdon, S. J. An intramolecular switch regulates phosphoindependent FHA domain interactions in Mycobacterium tuberculosis. Science signaling 2:ral2; 2009.
[34] Anand, N.; Singh, P.; Sharma, A.; Tiwari, S.; Singh, V.; Singh, D. K.; Srivastava, K. K.;
Singh, B. N.; Tripathi, R. P. Synthesis and evaluation of small libraries of triazolylmethoxy chalcones, flavanones and 2-aminopyrimidines as inhibitors of mycobacterial FAS-II and PknG. Bioorganic & medicinal chemistry; 2012.
[35] Ortiz-Lombardia, M.; Pompeo, F.; Boitel, B.; Alzari, P. M. Crystal structure of the catalytic domain of the PknB serine/threonine kinase from Mycobacterium tuberculosis. The Journal of biological chemistry 278:13094-13100; 2003.
[36] Bonacci, G.; Schopfer, F. J.; Batthyany, C. I.; Rudolph, T. K.; Rudolph, V.; Khoo, N. K.; Kelley, E. E.; Freeman, B. A. Electrophilic fatty acids regulate matrix metalloproteinase activity and expression. The Journal of biological chemistry 286:16074-16081; 2011.
[37] Sarver, A.; Scheffler, N. K.; Shetlar, M. D.; Gibson, B. W. Analysis of peptides and proteins containing nitrotyrosine by matrix-assisted laser desorption/ionization mass spectrometry. J Am Soc Mass Spectrom 12:439-448; 2001.
[38] Desper, R.; Gascuel, 0. Getting a tree fast: Neighbor Joining, FastME, and distance-based methods. Curr Protoc Bioinformatics Chapter 6:Unit 6 3; 2006.

What is claimed is:

1. Method of mediating regulatory role of kinase activity, comprising the step of administering to a subject in need thereof a nitrated fatty acid.

2. The method of claim 1, further comprising the step of causing iron loss in PknG from Mycobacterium tuberculosis.

3. The method of claim 1, wherein the nitrated fatty acid is nitrooleic acid.

4. The method of claim 3, wherein the nitrated fatty acid is selected from the group consisting of 9-nitrooleic acid, 10-nitrooleic acid or combinations thereof.

5. The method of claim 4, further comprising the step of nitroalkylation of a redox-sensitive non-catalytic domain of PknG in Mycobacterium tuberculosis.

6. Method of regulating PknG from Mycobacterium tuberculosis, comprising the step of administering a nitrated fatty acid to a subject in need thereof.

7. The method of claim 6, further comprising the step of selectively inhibits rubredoxin-containing enzymes with the nitrated fatty acid.

8. The method of claim 7, further comprising the step of inducing iron loss from the PknG protein.

9. The method of claim 6, further comprising the step of selectively inhibiting PknG phosphorylation.

10. The method of claim 9, further comprising the step of regulating GarA.


SEQUENCE LISTING (14 sequences)

SEQ ID NO: 1 - protein, 7 aa, Escherichia coli
FCWNCGR

SEQ ID NO: 2 - protein, 11 aa, Escherichia coli
FCWNCGRPVGR

SEQ ID NO: 3 - protein, 33 aa, Escherichia coli
GASEGWCPYCGSPYSFLPQLNPGDIVAGQYEVK

SEQ ID NO: 4 - protein, 18 aa, Escherichia coli
GLVHSGDAEAQAMAMAER

SEQ ID NO: 5 - protein, 24 aa, Escherichia coli
STFGVDLLVAHTDVYLDGQVHAEK

SEQ ID NO: 6 - protein, 23 aa, Escherichia coli
HGALDADGVDFSESVELPLMEVR

SEQ ID NO: 7 - protein, 16 aa, Escherichia coli
HFTEVLDTFPGELAPK

SEQ ID NO: 8 - protein, 6 aa, Escherichia coli
HFTTAR

SEQ ID NO: 9 - protein, 17 aa, Escherichia coli
ASTNHILGFPFTSHGLR

SEQ ID NO: 10 - protein, 11 aa, Escherichia coli
HRYTLVDMANK

SEQ ID NO: 11 - protein, 23 aa, Escherichia coli
QFLAEVVHPSIVQIFNFVEHTDR

SEQ ID NO: 12 - protein, 5 aa, Escherichia coli
  misc_feature (2)..(3): Xaa can be any naturally occurring amino acid
CXXCG

SEQ ID NO: 13 - protein, 17 aa, Escherichia coli
GCIAHGGLGWIYLALDR

SEQ ID NO: 14 - protein, 4 aa, Escherichia coli
CWNC